(19) United States
(12) Patent Application Publication
(10) Pub. No.: US 2024/0016540 A1
(43) Pub. Date: Jan. 18, 2024

(54) TIMING OF PULSED FIELD ABLATION ENERGY DELIVERIES

(71) Applicant: MEDTRONIC, INC, Minneapolis, MN (US)

(72) Inventors: Daniel C. Sigg, Saint Paul, MN (US); Lars M. Mattison, St. Anthony, MN (US); Damijan Miklavcic, Ljubljana (SI)

(21) Appl. No.: 18/338,046

(22) Filed: Jun. 20, 2023

Related U.S. Application Data
(60) Provisional application No. 63/368,233, filed on Jul. 12, 2022.

(51) Int. Cl. A61B 18/14; A61B 5/00

(52) U.S. Cl. CPC A61B 18/1492; A61B 5/0006; A61B 2018/00577

(57) ABSTRACT

Methods of and systems for ablating cardiac tissue is disclosed. One example method includes monitoring an electrical signal of a heart of a patient. The electrical signal represents the heart beating. The method further includes determining, with an electronic processor and based on the electrical signal, an end-diastolic time period at an end of a diastolic time period during which diastole of the heart has occurred during a previous cardiac cycle. The method further includes determining, with the electronic processor and based on the electrical signal, that another cardiac cycle has begun. The method further includes causing, with the electronic processor, an electrode to deliver pulsed field ablation (PFA) energy to the heart during at least a portion of a time in which the end-diastolic time period of the another cardiac cycle is expected to occur.

### FIELD

The present technology is generally related to methods and systems for treating tissue with electroporation or pulsed-field ablation.

### BACKGROUND

There are many medical treatments that involve instances of cutting, ablating, coagulating, destroying, or otherwise changing the physiological properties of tissue. These techniques can be used beneficially to change the electrophysiological properties of tissue, such as those associated with cardiac arrhythmias or other electrophysiological abnormalities. In particular, normal sinus rhythm of the heart begins with the sinoatrial node (“SA node”) generating a depolarization wave front that causes adjacent myocardial tissue cells in the atria to depolarize. The depolarization propagates across the atria, causing the atria to contract and empty blood from the atria into the ventricles. The impulse is next delivered via the atrioventricular node (“AV node”) and via the HIS-Purkinje system to myocardial tissue cells of the ventricles. The depolarization of cells propagates across the ventricles, causing the ventricles to contract. This conduction system results in the described, organized sequence of myocardial contraction leading to a normal heartbeat.

Sometimes, anatomical obstacles such as fibrosis, fibrotic scar, or uneven distribution of refractoriness of cardiac myocytes in certain parts of the heart in the atria or ventricles can lead to aberrant conductive pathways in heart tissue that disrupt the normal path of depolarization events. These anatomical obstacles or “conduction blocks” can cause the electrical impulse to degenerate into several circulating wavelets that circulate about the obstacles. The aberrant conductive pathways create abnormal, irregular, and sometimes life-threatening heart rhythms called arrhythmias. An arrhythmia can take place in the atria, for example, as in atrial tachycardia, atrial fibrillation (“AF”), or atrial flutter. The arrhythmia can also take place in the ventricle, for example, as in ventricular tachycardia. Additionally, there may be ectopic sites within the heart that produce premature activations from such tissue sites, producing arrhythmogenic conduction patterns. For example, ectopic sites within the pulmonary veins are one of the key mechanisms of induction and maintenance of atrial fibrillation.

One approach to treating an arrhythmia includes creating one or more lesions that compartmentalize the aberrant pathway and direct electrical conduction along selected pathways to promote organized signal conduction, while also isolating AF triggers from connecting with the atria. Often, the application of energy is used to destroy cells at the ablation site while leaving the surrounding structures of the organ largely intact. Radiofrequency (“RF”) energy and cryogenic cooling have been found to be highly viable in this regard, and are commonly employed. Other ablative techniques include the application of ultrasound, microwave, laser, cytotoxic agents (e.g., alcohol), etc. Yet another ablative technique includes applying energy in the form of pulsed electrical fields (PEF), which also may be referred to as pulsed field ablation (PFA).

### SUMMARY

Pulsed field ablation (PFA) is a term used to explain an application of energy in the form of pulsed electric fields (PEFs) to ablate cardiac tissues (i.e., creating lesions) via mechanisms of electroporation (e.g., irreversible electroporation and reversible electroporation). Electric fields and lesions created by the electric fields may be dependent on many factors including, but not limited to, applied voltage, electrode configuration, pulse wave form, pulse trains, and proximity of electrode to target tissue.

The techniques of this disclosure generally relate to timing of PFA energy deliveries based on a cardiac cycle to control (and increase or maximize) lesion depth and transmurality achieved by the pulsed field ablation. Specifically, PFA energy may be delivered during a point of a cardiac cycle during diastole (and particularly during an end-diastolic time period) for respective specific areas of the heart at which PFA energy is being delivered (via electrodes on a catheter) to increase or maximize lesion depth and/or thus achieve transmural lesions.

In one example, the present disclosure provides a method of ablating cardiac tissue. The method may include monitoring an electrical signal of a heart of a patient. The electrical signal represents the heart beating. The method may further include determining, with an electronic processor and based on the electrical signal, an end-diastolic time period at an end of a diastolic time period during which diastole of the heart has occurred during a previous cardiac cycle. The method may further include determining, with the electronic processor and based on the electrical signal, that another cardiac cycle has begun. The method may further include causing, with the electronic processor, an electrode to deliver pulsed field ablation (PFA) energy to the heart during at least a portion of a time in which the end-diastolic time period of the another cardiac cycle is expected to occur.

In another aspect, determining the end-diastolic time period includes determining, with the electronic processor and based on the electrical signal, a first time interval between occurrences of a first wave and a second wave included in the electrical signal of one or more previous cardiac cycles.

In another aspect, determining that the another cardiac cycle has begun includes determining, with the electronic processor and based on the electrical signal, that another instance of the first wave has occurred in a current cardiac cycle.

In another aspect, causing the electrode to deliver the PFA energy to the heart includes causing, with the electronic processor, the electrode to deliver the PFA energy to the heart during the at least a portion of the time in which the end-diastolic time period of the current cardiac cycle is expected to occur based on the first time interval.

In another aspect, the first wave and the second wave are the same type of wave, and the first time interval occurs between successive occurrences of a first type of wave included in the electrical signal.

In another aspect, the first time interval includes an RR interval and the first type of wave includes an R-wave.

In another aspect, the method further includes selecting a type of the first type of wave based on a location of a treatment site of the heart that is intended to receive the PFA energy.

In another aspect, the first wave is a first type of wave and the second wave is a second type of wave that is different than the first type of wave.

In another aspect, the first type of wave includes a P-wave and the second type of wave includes an R-wave, and the first time interval includes a PR interval.

In another aspect, the method further includes selecting the first type of wave, the second type of wave, or both based on a location of a treatment site of the heart that is intended to receive the PFA energy.

In another aspect, determining the first time interval includes determining a time value for each of a plurality of first time intervals included in the electrical signal over an evaluation time period before the PFA energy is delivered; determining a variation between the time values; comparing the variation to a variation threshold; and in response to determining that the variation is below the variation threshold, establishing the first time interval by determining an average of the time values.

In another aspect, the evaluation time period is longer when the heart of the patient is not artificially paced than when the heart of the patient is artificially paced.

In another aspect, the method further includes determining, with the electronic processor, that the end-diastolic time period is expected to occur during a time in a range of 90% to 99% of the first time interval after the another instance of the first type of wave occurred.

In another aspect, the diastole of the heart includes one of diastole of a left ventricle, diastole of a right ventricle, diastole of a left atrium, and diastole of a right atrium.

In another aspect, the end-diastolic time period of the another cardiac cycle indicates that a treatment site of the heart that is intended to receive the PFA energy includes myocardium that has a minimum thickness, compared to a thickness of the myocardium throughout the rest of the another cardiac cycle, during at least a portion of the time in which the end-diastolic time period of the another cardiac cycle is expected to occur.

In another embodiment, the present disclosure provides a system for ablating cardiac tissue. The system may include a generator including an electronic processor that may be configured to monitor an electrical signal of a heart of a patient. The electrical signal represents the heart beating. The electronic processor may be further configured to determine, based on the electrical signal, an end-diastolic time period at an end of a diastolic time period during which diastole of the heart has occurred during a previous cardiac cycle. The electronic processor may be further configured to determine, based on the electrical signal, that another cardiac cycle has begun. The electronic processor may be further configured to cause an electrode to deliver pulsed field ablation (PFA) energy to the heart during at least a portion of a time in which the end-diastolic time period of the another cardiac cycle is expected to occur.

In another aspect, the electronic processor is configured to determine the end-diastolic time period by determining, based on the electrical signal, a first time interval between occurrences of a first wave and a second wave included in the electrical signal of one or more previous cardiac cycles.

In another aspect, the electronic processor is configured to determine that the another cardiac cycle has begun by determining, based on the electrical signal, that another instance of the first wave has occurred in a current cardiac cycle.

In another aspect, the electronic processor is configured to cause the electrode to deliver the PFA energy to the heart by causing, the electrode to deliver the PFA energy to the heart during the at least a portion of the time in which the end-diastolic time period of the current cardiac cycle is expected to occur based on the first time interval.

In another aspect, the electronic processor is configured to determine that the end-diastolic time period is expected to occur during a time in a range of 90% to 99% of the first time interval after the another instance of the first type of wave occurred.

In another aspect, the diastole of the heart includes one of diastole of a left ventricle, diastole of a right ventricle, diastole of a left atrium, and diastole of a right atrium.

In another aspect, the end-diastolic time period of the another cardiac cycle indicates that a treatment site of the heart that is intended to receive the PFA energy includes myocardium that has a minimum thickness, compared to a thickness of the myocardium throughout the rest of the another cardiac cycle, during at least a portion of the time in which the end-diastolic time period of the another cardiac cycle is expected to occur.

In another example, the present disclosure provides a method of ablating cardiac tissue. The method may include monitoring an electrical signal of a heart of a patient. The electrical signal represents the heart beating. The method may further include determining, with an electronic processor and based on the electrical signal, a first time interval between occurrences of a first wave and a second wave included in the electrical signal of one or more previous cardiac cycles. The method may further include determining, with the electronic processor and based on the electrical signal, that another instance of the first wave has occurred in a current cardiac cycle. The method may further include causing, with the electronic processor, an electrode to deliver pulsed field ablation (PFA) energy to the heart during at least a portion of a time included in a range of 90% to 99% of the first time interval after the first wave has occurred in the current cardiac cycle.

In another aspect, the time included in the range of 90% to 99% of the first time interval after the first wave has occurred indicates that a treatment site of the heart that is intended to receive the PFA energy includes myocardium that has a minimum thickness, compared to a thickness of the myocardium throughout the rest of the current cardiac cycle, during at least a portion of the time included in the range of 90% to 99% of the first time interval after the first wave has occurred.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 shows an example ablation system including a pulsed-field ablation device having an example distal curvilinear electrode array portion according to one example.

FIG. 2 is a block diagram of the generator of the of the ablation system of FIG. 1 according to one example.

FIG. 3 illustrates numerous graphs of different known measured values and characteristics of an example heart throughout example known cardiac cycles (i.e., heart beats) according to one example.

FIGS. 4A and 4B illustrate simplified diagrams of an example electric field being applied to myocardium of different thicknesses according to one example.

FIG. 5 illustrates a flowchart of a method performed by an electronic processor of the generator of FIG. 2 to control delivery of pulsed-field ablation energy to a heart of a patient.

### DETAILED DESCRIPTION

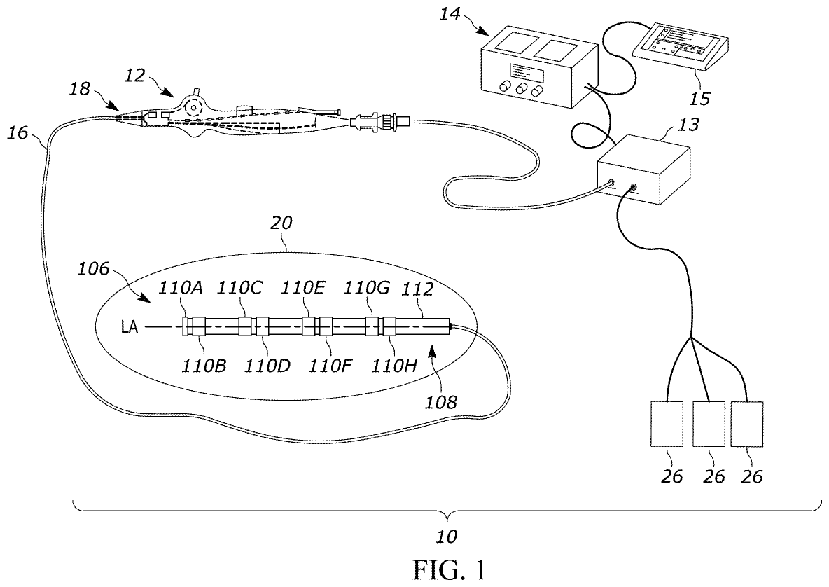

The present application provides methods and systems for diagnosing and/or treating undesirable physiological or anatomical tissue regions, such as those contributing to aberrant electrical pathways in the heart. Referring now to the figures in which like reference designations refer to like elements, an example of a medical system constructed in accordance with principles of the present invention is shown in FIG. 1 and generally designated as “10.” The system 10 generally includes a medical device 12 that may be coupled directly to an energy supply, for example, a pulse field ablation (PFA) generator 14 including an energy control, delivering system, and a monitoring system. In some aspects, the medical device 12 may be coupled to the energy supply indirectly through a catheter electrode distribution system 13. A remote controller 15 may further be included in communication with the generator 14 for operating and controlling the various functions of the generator 14. The medical device 12 may generally include one or more diagnostic or treatment regions for energetic, therapeutic, and/or investigatory interaction between the medical device 12 and a treatment site. The treatment region(s) may deliver, for example, pulsed electroporation energy to a tissue area in proximity to the treatment region(s). One or more of the devices shown in FIG. 1 may be combined into a single device. For example, the catheter electrode distribution system/box 13 may not be present and its functionality may be combined into a single system/box with the generator 14 and/or the remote controller 15.

The medical device 12 may include an elongate body 16 passable through a patient's vasculature and/or positionable proximate to a tissue region for diagnosis or treatment, such as a catheter, sheath, or intravascular introducer. The elongate body 16 may define a proximal portion 18 and a distal portion 20, and may further include one or more lumens disposed within the elongate body 16 thereby providing mechanical, electrical, and/or fluid communication between the proximal portion 18 of the elongate body 16 and the distal portion 20 of the elongate body 16. The distal portion 20 may generally define the one or more treatment region(s) of the medical device 12 that are operable to monitor, diagnose, and/or treat a portion of a patient. The treatment region(s) may have a variety of configurations to facilitate such operation. In some instances, distal portion 20 (which is shown in enlarged scale in FIG. 1) includes catheter/elongated structure 112 carrying a plurality of electrodes 110A-110H (collectively, “electrodes 110”). Catheter 112 may include a distal portion 106 and a proximal portion 108. Electrodes 110 may be generally positioned at distal portion 106, while proximal portion 108 may be ultimately connected to the catheter electrode distribution system 13. The electrodes 110 may be configured to deliver pulsed-field energy. For example, the generator 14 (e.g., an electronic processor 205 of the generator 14) may cause one or more of the electrodes 110 to provide/deliver PFA energy using one or more different modes such as a focal mode. For instance, to operate the electrodes 110 in the focal mode, energy may be output to the electrodes 110 by the generator 14 to cause the electrodes 110 to generate a field with a geometry focused at the distal portion/tip 106 of catheter 112 (e.g., focused at tip electrode 110A). Such a focused field geometry may result in lesions forming proximal to the tip 106 of the catheter 112. As used herein, causing an electrode(s) 110 to provide/deliver PFA energy means controlling the generator 14 (e.g., the electronic processor 205 of the generator 14) to generate waveforms that are transmitted using the catheter 112 to the electrode(s) 110 to provide/deliver PFA energy via the electrode(s) 110 as discussed herein. Electrodes 110 may be of any suitable geometry. Example geometries of electrodes include, but are not necessarily limited to, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes). Electrodes 110 may be axially distributed along longitudinal axis LA of the catheter 112. The catheter 112 and the electrodes 110 shown in FIG. 1 are merely examples. In some instances, the catheter 112 may include more or less electrodes 112. Additionally or alternatively, the electrodes 110 may be arranged in different configurations including using coils or other return electrodes. In some instances, the catheter 112 may be a different shape at a point where the catheter 112 contacts tissue of the patient. The system may also be designed to vector the energy in a unipolar configuration (between electrodes 110 and return patches located on skin of patient (not shown)). Further, the system may also be designed to vector energy in a two catheter configuration (between one or any number of electrodes 110) and another indwelling catheter with return electrodes (not shown).

The system 10 may further include three or more electrocardiogram (ECG) electrodes 26 configured to be placed in or on the patient and configured to be in communication with the generator 14 through the catheter electrode distribution box 13 to monitor the patient's cardiac activity for use in determining pulse train delivery timing at the desired portion of the cardiac cycle as explained in greater detail below. In addition to monitoring, recording, or otherwise conveying measurements or conditions within the medical device 12 or the ambient environment at the distal portion of the medical device 12, additional measurements may be made through connections to the multi-electrode catheter including for example temperature, electrode-tissue interface impedance, delivered charge, current, power, voltage, work, or the like in the generator 14 and/or the medical device 12. The surface ECG electrodes 26 may be in communication with the generator 14 for initiating or triggering one or more alerts or therapeutic deliveries during operation of the medical device 12. In some instances, additional or alternative information is used to monitor patient information such as cardiac cycle and timing. For example, the system 10 may receive intracardiac electrogram (EGM) information and/or other information from one or more other electrodes and/or devices/sensors. Additional neutral electrode patient ground patches (not pictured) may be employed to evaluate the desired bipolar electrical path impedance, as well as monitor and alert the operator upon detection of inappropriate and/or unsafe conditions, which include, for example, improper (either excessive or inadequate) delivery of charge, current, power, voltage and work performed by the plurality of electrodes 110; improper and/or excessive temperatures of the plurality of electrodes 110; improper electrode-tissue interface impedances; improper and/or inadvertent electrical connection to the patient prior to delivery of high voltage energy by delivering one or more low voltage test pulses to evaluate the integrity of the tissue electrical path.

The generator 14 may include an electrical current or pulse generator having a plurality of output channels, with each channel coupled to an individual electrode of the plurality of electrodes 110 or multiple electrodes of the plurality of electrodes 110 of the medical device 12. The generator 14 may be operable in one or more modes of operation, including for example: (i) bipolar energy delivery between at least two electrodes 110 or electrically-conductive portions of the medical device 12 within a patient's body, (ii) monopolar or unipolar energy delivery to one or more of the electrodes 110 or electrically-conductive portions on the medical device 12 within a patient's body and through either a second device (e.g., catheter) within the body (not shown) or a patient return or ground electrode (not shown) spaced apart from the plurality of electrodes 110 of the medical device 12, such as on a patient's skin or on an auxiliary device positioned within the patient away from the medical device 12, for example, and (iii) a combination of the monopolar and bipolar modes.

The generator 14 may provide electrical pulses to the medical device 12 to perform an electroporation procedure to cardiac tissue or other tissues within the body, for example, excitable tissue such as skeletal/smooth muscle tissue. “Electroporation” utilizes high amplitude pulses to effectuate a physiological modification (i.e., permeabilization) of the cells to which the energy is applied. Such pulses may preferably be short (e.g., nanosecond, microsecond, or millisecond pulse width) in order to allow application of high voltage, high current (for example, 20 or more amps) without long duration of electrical current flow that results in significant tissue heating and neuromuscular stimulation (e.g., phrenic nerve). In particular, the pulsed energy induces the formation of microscopic pores or defects in the cell membrane. Depending upon the characteristics of the electrical pulses, an electroporated cell can survive electroporation (i.e., “reversible electroporation”) or die (i.e., irreversible electroporation, “IEP”). Reversible electroporation may be used to transfer agents, including large molecules, into targeted cells for various purposes, including alteration of the action potentials of cardiac myocytes. The methods, systems, and devices described herein may be used to perform irreversible electroporation or reversible electroporation.

The generator 14 may be configured and programmed to deliver pulsed, high voltage electric fields appropriate for achieving desired pulsed, high voltage ablation (or pulsed field ablation). As a point of reference, the pulsed, high voltage, non-radiofrequency, ablation effects of the present disclosure are distinguishable from DC current ablation, as well as thermally-induced ablation attendant with conventional RF techniques. For example, the pulse trains delivered by generator 14 are delivered at a lower frequency than radiofrequency treatments. The pulsed-field energy in accordance with the present disclosure is sufficient to induce cell death for purposes of completely blocking an aberrant conductive pathway along or through cardiac tissue, destroying the ability of the so-ablated cardiac tissue to propagate or conduct cardiac depolarization waveforms and associated electrical signals.

The plurality of electrodes 110 may also perform diagnostic functions such as collection of intracardiac electrograms (EGM) as well as performing selective pacing of intracardiac sites for diagnostic purposes. In one configuration, the measured ECG signals are transferred from the catheter electrode energy distribution system 13 to an electrophysiology (EP) recording system input box (not shown) that is included with generator 14. The plurality of electrodes 110 may also monitor the proximity to target tissues and quality of contact with such tissues using impedance based measurements with connections to the catheter electrode energy distribution system 13. The catheter electrode energy distribution system 13 may include high speed relays to disconnect/reconnected specific electrode 110 from the generator 14 during therapies. Immediately following the pulsed energy deliveries, the relays reconnect the electrodes 110 so they may be used for diagnostic purposes.

In some aspects, the plurality of electrodes 110 may deliver therapeutic biphasic pulses having a preprogrammed pattern and duty cycle as explained in U.S. Pat. No. 10,531,914, which is incorporated by reference. In some aspects, a pulse train when delivered from a bipolar electrode array (such as the array shown in FIG. 1) may produce lesions in cardiac muscle in the range of approximately 2-3 millimeters deep, 4-7 millimeters deep, and/or the like. Altering parameters of the pulse train (e.g., voltage, current, pulse width, number of pulses per pulse train, number of pulse trains, etc.) influences lesion size. For example, increased voltage may correspondingly increase the lesion depth.

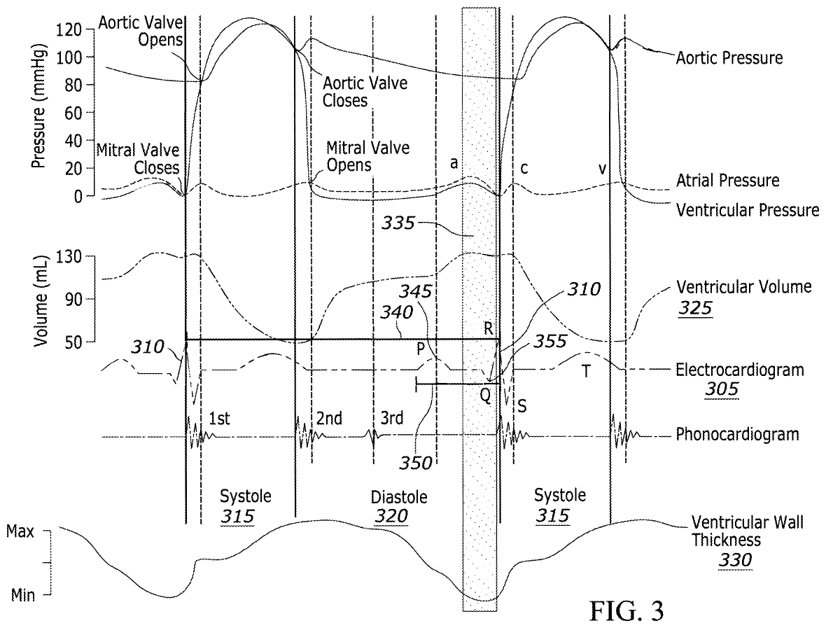

As explained previously herein, the system 10 may include ECG electrodes 26 electrically couplable to the generator 14 and configured to measure electrical signals from the heart. The ECG measurements made by the ECG electrodes 26 may be sequentially or simultaneously made with the delivery of the pulse trains from the plurality of electrodes 110. In an example configuration, three ECG electrodes 26 are adhered the surface of the patient and are further coupled to the generator 14. The generator 14 may be configured to process and correlate the measured Einthoven signals into a determination of when to deliver pulses as explained in greater detail below. For example, the generator 14 may be programmed with predetermined measured patient parameters, for example, timing and amplitude parameters associated with a QRS wave (see graph 330 of FIG. 3) to control timing of the delivery of PFA energy to a treatment site of the heart of the patient as explained in greater detail below. When at least one of the predetermined measured patient parameters are met, the generator 14 may initiate the delivery of pulses for a predetermined period of time.

The pulsed field of energy may be delivered in a bipolar fashion, between odd and even electrodes, in monophasic or biphasic pulses. The application of bipolar biphasic electrical pulses may produce beneficial results in the context of cardiac tissue ablation such as a well-controlled dimension/shape of the electrical field and less neuromuscular stimulation. With biphasic electroporation pulses, the direction of the pulses completing one cycle alternates in a few microseconds. As a result, the cells to which the biphasic electrical pulses are applied undergo alternation of electrical field bias. Changing the direction of bias reduces prolonged post-ablation depolarization and/or ion charging. As a result, prolonged muscle excitation (e.g., skeletal and cardiac cells) and risks of post shock fibrillation of the cardiac cells may be reduced. Additional details of example pulses/pulse trains that may be used during PFA are explained in U.S. Pat. No. 10,531,914, which is incorporated by reference.

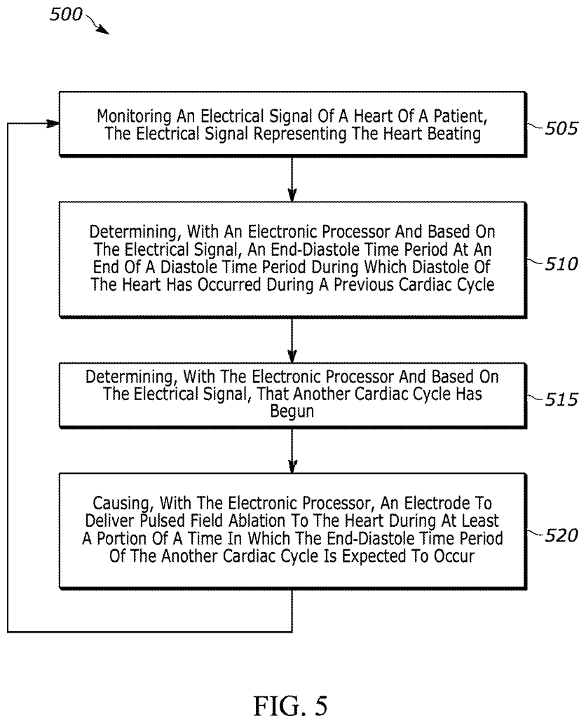

In some instances, the generator 14 controls timing of PFA energy deliveries to a treatment site (e.g., a specific area of a heart) via one or more electrodes 110 based on a cardiac cycle to control (and increase or maximize) lesion depth and transmurality achieved by the by the PFA. Specifically, PFA energy may be delivered during a point of a cardiac cycle during diastole (and particularly during an end-diastolic time period) for respective specific areas of the heart at which PFA energy is being delivered (via electrodes 110 on the catheter 112) to increase or maximize lesion depth and/or thus achieve transmural lesions. For example, in some instances, the generator 14 may perform a method 500 shown in FIG. 5 to control delivery of PFA energy to a treatment area by controlling generation of waveforms/signals provided to the electrode(s) 110 to cause the electrode(s) 110 to provide/deliver PFA energy. In some instances, a diastolic time period of a specific area of the heart is a phase of the heartbeat when the heart muscle relaxes and allows chambers of the heart to fill with blood (e.g., see diastolic time period 320 in FIG. 3). In some instances, an end-diastolic time period is a time period at the end of the diastolic time period. For example, the end-diastolic time period may be just before occurrence of a peak of an electrical stimulus wave (e.g., an R-wave) that starts contraction (i.e., a systolic time period) of the specific area of the heart (e.g., see ventricular end-diastolic time period 335 of FIG. 3). The contraction of the heart is as a systole phase of the heartbeat when the heart muscle contracts and pumps blood from the chambers of the heart into arteries. In some instances, the end-diastolic time period may occur during a range of 90% to 99.99% (or 80% to 99.99% or 95% to 99.99% or the like) of the diastolic time period.

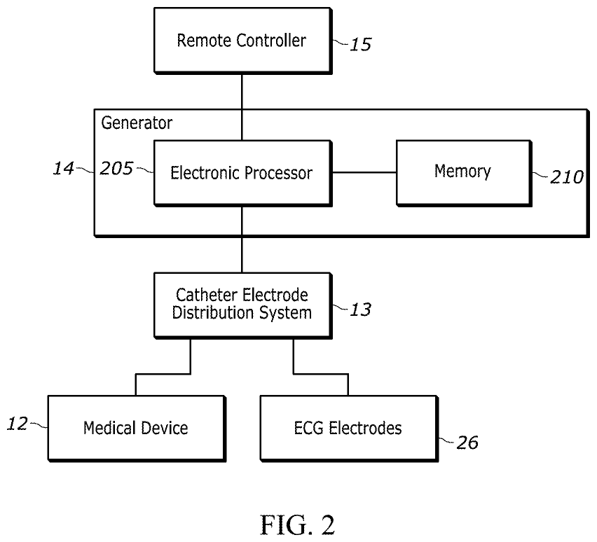

FIG. 2 is a block diagram of the generator 14 of the ablation system 10 according to one example. In the example shown, the generator 14 includes an electronic processor 205 (for example, a microprocessor or another electronic device). The electronic processor 205 may be electrically connected to a memory 210 and may include input and output interfaces to couple with other devices of the system 10, for example, the remote controller 15 and the catheter electrode distribution system 13 as shown in FIG. 2.

The memory 210 may include read only memory (ROM), random access memory (RAM), other non-transitory computer-readable media, or a combination thereof. The electronic processor 205 is configured to receive instructions and data from the memory 210 and execute, among other things, the instructions. In particular, the electronic processor 205 executes instructions or algorithms stored in the memory 210 to provide for the automated operation and performance of the features, sequences, calculations, or procedures described herein.

In some aspects, the generator 14 may include fewer or additional components in configurations different from that illustrated in FIG. 2. For example, in some aspects, the generator 14 includes one or more additional electronic processors that may perform specific functions and that may be communicatively coupled to each other and/or to the electronic processor 205. As another example, the generator 14 may include a display and/or an integrated user input device in addition to or as an alternative to the remote controller 15.

Other devices of the system 10 may include similar components as the generator 14. For example, the catheter electrode distribution system 13, the remote controller 15, and/or the medical device 12 may each include an electronic processor and a memory similar to those described previously herein with respect to the generator 14. In some aspects, these other devices 12, 13, 15 may additionally or alternatively have other components that allow each device 12,13, 15 to perform its respective functionality as described herein.

In some instances, the electronic processor 205 of the generator 14 controls timing of PFA energy deliveries to a treatment site (e.g., a specific area of a heart) via one or more electrodes 110 based on a cardiac cycle to control (and increase or maximize) lesion depth and transmurality achieved by the PFA. Specifically, PFA energy may be delivered during a point of a cardiac cycle during diastole (and particularly during an end-diastolic time period) for respective specific areas of the heart at which PFA energy is being delivered (via electrodes 110 on the catheter 112) to increase or maximize lesion depth and/or thus achieve transmural lesions. For example, in some instances, the electronic processor 205 may perform a method 500 shown in FIG. 5 to control delivery of PFA energy to a treatment area by controlling generation of waveforms/signals provided to the electrode(s) 110 to cause the electrode(s) 110 to provide/deliver PFA energy.

Additionally or alternatively, in some instances, the electronic processor 205 may control delivery of PFA energy to a treatment area such that PFA energy is delivered at times when cardiomyocytes in the treatment area are aligned/oriented in a manner that allows for greater levels of ablation (e.g., more effective ablation) compared to other alignments/orientations of the cardiomyocytes. For example, twisted anisotropy may be present in ventricles or other treatment areas such that the alignment/orientation of cardiomyocytes changes during the heartbeat. Based on certain alignments/orientations of cardiomyocytes of the treatment area during different time periods of the heartbeat (e.g., as determined by monitoring an individual patient or as determined in general through research and monitoring of many patients), the electronic processor 205 may control delivery of PFA energy to the treatment area to occur at times when cardiomyocytes in the treatment area are aligned/oriented in a manner that allows for greater levels of ablation compared to other alignments/orientations of the cardiomyocytes (e.g., PFA energy delivered when many cardiomyocytes are aligned in a same or similar direction). Additional information regarding how cell orientation of a treatment area may affect ablation of the treatment area using some types of PFA energy is disclosed in U.S. patent application Ser. No. 18/295,416, filed Apr. 4, 2023, which is incorporated by reference.

FIG. 3 illustrates numerous graphs of different known measured values and characteristics of an example heart throughout example known cardiac cycles (i.e., heart beats). For example, FIG. 3 illustrates an example QRS complex in a graph 305 of an electrocardiogram (ECG). The QRS complex includes an R-wave 310 that indicates depolarization of the main mass of the ventricles of a heart to cause contraction of the ventricles to force blood out of the heart. A ventricular systolic time period 315 and a ventricular diastolic time period 320 are also labeled in FIG. 3. The systolic time period 315 may indicate a phase of a cardiac cycle when the ventricular heart muscle contracts to pump blood from the chambers of the heart into the arteries. The diastolic time period 320 may indicate a phase of the cardiac cycle when the ventricular heart muscle relaxes and allows the chambers of the heart to fill with blood. The graph 305 of the ECG includes labels for other well-known wave types of a cardiac cycle (e.g., P-wave 345, T-wave, Q-wave 355, and S-wave).

Among other graphs, FIG. 3 also includes a graph 325 of ventricular volume during a cardiac cycle and a graph 330 of ventricular wall thickness (i.e., myocardium thickness) during a cardiac cycle. The graph 330 specifically indicates left ventricle wall thickness. As shown in FIG. 3, the graphs 325 and 330 are approximately inversely related to each other at many points of the cardiac cycle. For example, just before and during the occurrence of an R-wave 310 which transitions the ventricles of the heart from the diastolic time period 320 to the systolic time period 315, the ventricular volume is at a maximum, and the ventricular wall thickness is at a minimum. Conversely, after blood is forced out of the heart and when the ventricles of the heart transition back to the diastolic time period 320 from the systolic time period 315 to begin filling back up with blood, the ventricular volume is at a minimum, and the ventricular wall thickness is at a maximum.

FIG. 3 illustrates numerous graphs that correspond to one of the example situations described herein when the electrodes 110 are located on a left ventricle of the heart to deliver PFA to the left ventricle. However, as indicated herein, the electrodes 110 may be placed at other areas of the heart to deliver PFA such as one of the atria. Characteristics of the atria of the heart (e.g., volume, wall thickness, etc.) may change differently than those same characteristics of the ventricles. FIG. 3 mostly shows graphs corresponding to characteristics of the ventricles. However, values for the same characteristics may be determined and/or estimated for the example heart throughout example known cardiac cycles when the electrodes 110 are used to deliver PFA to other areas of the heart. For example, in situations where the electrodes 110 are used to deliver PFA to the atria, the characteristics of the atria are used to control timing of PFA delivery instead of using characteristics of the ventricles. Accordingly, the systolic and diastolic time periods 315, 320 labeled in FIG. 3 may be different sizes and/or may be shifted to be located in different time periods of the cardiac cycle when the electrodes 110 are used to deliver PFA to other areas of the heart such as the atria because other areas of the heart have different systolic and diastolic time periods than the ventricles. While FIG. 3 does not include a graph of the atrial wall thickness, such a graph (and/or data corresponding thereto) may be referenced/used by the electronic processor 205 in situations where the electrodes 110 are used to deliver PFA to the atria.

As indicated previously herein, electric fields and lesions created by the electric fields during pulsed field ablation (PFA) may be dependent on many factors. Given the high voltages/currents applied during PFA, there is a concern of potentially creating arrhythmogenesis. Accordingly, some PFA systems are R-wave gated such that these systems deliver pulses during the ventricular systolic time period 315 (i.e., during maximum contractions of ventricular cardiac muscles). In other words, in existing systems, PFA energy is delivered during a time period immediately following a detection of an R-wave 310 in a patient's cardiac cycle (e.g., during an early part of the ventricular systolic time period 315) that is indicative of depolarization of the main mass of the ventricles of a heart.

However, delivering PFA energy during the ventricular systolic time period 315 immediately following detection of an R-wave 310 is not effective in maximizing lesion depth. In fact, in particular in the ventricles, the thickness of the myocardium is at a high level (often its maximum) during systole (i.e., the time period immediately following a detection of an R-wave 310). For example, the graph 330 of FIG. 3 indicates that the ventricular wall thickness is high (i.e., at or nearing its maximum) at the end of the ventricular systolic time period 315 when the ventricular heart muscles have just contracted and at the beginning of the ventricular diastolic time period 320 before the heart muscles have fully relaxed to let blood enter the heart. Additionally, the graph 330 of FIG. 3 indicates that the ventricular wall thickness is at approximately 50% of its maximum thickness or higher for almost all of the systolic time period 315. Accordingly, delivering PFA energy to a ventricle during the ventricular systolic time period 315 results in lower than maximum depth of penetration into the myocardium (and, in some instances, results in not achieving an intended depth of lesion or transmural lesion) compared to delivering PFA energy to the ventricle during other time periods of the cardiac cycle when the ventricular wall thickness is less (i.e., thinner). Similar issues exist when the PFA energy is delivered to other areas of the heart (e.g., one of the atria) although the timing of atrial systole and atrial diastole is different than ventricular systole and ventricular diastole. Thus, in some instances, PFA energy may be delivered to cardiac tissue at a sub-optimal time that does not maximize lesion depth.

To address the above-noted potential timing issue, a gated PFA train may be delivered during a diastolic time period (e.g., a ventricular diastolic time period 320) of a cardiac cycle. As shown in the graph 330 of FIG. 3, during ventricular diastole (and particularly during the end of the ventricular diastolic time period 320 just before the peak of the R-wave 310 at a ventricular end-diastolic time period 335), the ventricular cardiac muscle is relaxed, and ventricular wall thickness is thinner than the ventricular wall thickness during ventricular systole. In fact, as indicated in the graph 330 of FIG. 3, ventricular wall thickness is at a minimum during the ventricular end-diastolic time period 335 just before occurrence of the peak of the R-wave 310 that starts isometric contraction of the ventricles. Thus, delivery of PFA energy to a ventricle during ventricular diastole of a cardiac cycle (and particularly during the ventricular end-diastolic time period 335) results in increased penetration into the ventricular myocardium (i.e., increased lesion depth). In similar fashion, in situations where PFA energy is provided to one of the atria, the delivery of PFA energy may be timed in accordance with atrial diastole or an atrial end-diastolic period. For example, the delivery of PFA energy may be timed to be immediately before the contraction of the atria, (i.e., immediately before the P-wave) to optimize transmural lesion creation in the atria.

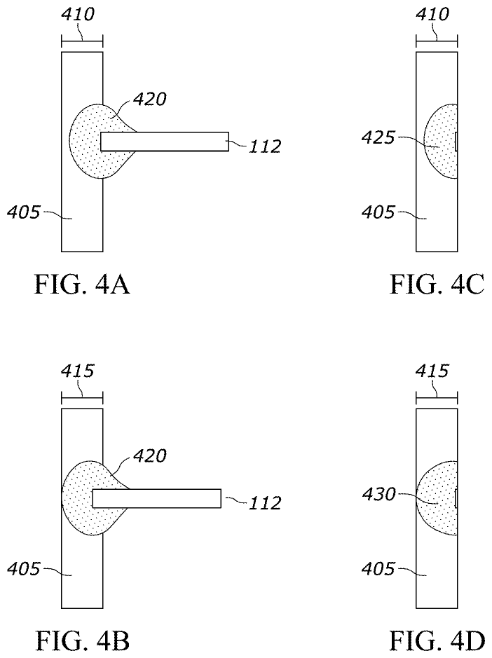

For example, FIGS. 4A and 4B illustrate simplified diagrams of the same electric field being applied to myocardium of different thicknesses (e.g., at different time periods in a cardiac cycle). FIGS. 4C and 4D illustrate simplified diagrams of an example lesion depth being achieved with the same electric field in the respective situations of FIGS. 4A and 4B. In FIG. 4A, myocardium 405 during systole (e.g., ventricular systole 315) has a first thickness 410 that is greater than a second thickness 415 that the myocardium has during diastole (e.g., ventricular diastole 320) (and particularly during the ventricular end-diastolic time period 335) as shown in FIG. 4B. In each of FIGS. 4A and 4B, the same electric field 420 is applied to the myocardium 405. In FIG. 4A, the electric field 420 is non-transmural as the electric field 420 does not penetrate the entire first thickness 410 of the myocardium 405. Accordingly, as shown in FIG. 4C, a corresponding depth of a lesion 425 (i.e., lesion depth/dimension) created by the electric field 420 is non-transmural (e.g., suboptimal). On the other hand, in FIG. 4B, the electric field 420 is transmural as the electric field 420 penetrates the entire second thickness 415 of the myocardium 405. Accordingly, as shown in FIG. 4D, a corresponding depth of a lesion 430 (i.e., lesion depth/dimension) created by the electric field 420 is transmural (i.e., existing or occurring across the entire wall of the myocardium 405).

Creating deeper lesions may be clinically relevant during either atrial or ventricular ablations. To date, there is little evidence that biphasic high frequency pulse wave forms used during PFA are arrhythmogenic, particularly when the PFA energy is delivered to a treatment area during the end-diastole time period 335 of FIG. 3. Accordingly, delivering PFA energy to atria or ventricles during respective atrial or ventricular diastole (and particularly during respective end-diastolic time periods such as the ventricular end-diastolic period 335 when PFA energy is being delivered to a ventricle) addresses the above-noted technological problem by (i) increasing lesion depth compared to delivering PFA energy during systole (e.g., immediately after detecting an R-wave 310 that indicates ventricular systole 315 when delivering PFA to a ventricle) (ii) without increasing the chances of creating arrhythmogenesis significantly or at all.

FIG. 5 illustrates a flowchart of a method 500 performed by the electronic processor of the generator 14 (possibly in conjunction with other devices in the system 100) to control delivery of PFA energy to a heart (cardiac tissue) of a patient. While a particular order of processing steps is indicated in FIG. 5 as an example, timing and ordering of such steps may vary where appropriate without negating the purpose and advantages of the examples set forth in detail throughout the remainder of this disclosure.

At block 505, an electrical signal of a heart of a patient is monitored. The electrical signal represents the heart beating. For example, an electrocardiogram (ECG) of the heart of the patient is determined by the electronic processor 205 of the generator 14. In some aspects, the ECG may be determined by another electronic processor of another device. The ECG is determined based on an electrical signal received from one or more electrodes. The electrodes that provide the electrical signal that allows for the ECG to be determined may include one or more of the electrodes 110, one or more of the ECG electrodes 26, or a combination thereof. In some aspects, a first electrode 110 that delivers PFA energy to a treatment site of the heart may also be used to monitor the electrical signal of the heart (e.g., the ECG). For example, unipolar signals may be measured from an indwelling PFA catheter with an electrode 110, and PFA energy may be delivered from the same electrode 110. As another example, bipolar signals may be measured from an indwelling PFA catheter from two electrodes 110, and PFA energy may be delivered in bipolar fashion from both electrodes 110. In some aspects, the two above-noted examples may be mixed and matched. Specifically, unipolar signals can be measured by an indwelling catheter in an aspect where bipolar PFA energy is delivered to the treatment site or vice versa. In some aspects, a second electrode (e.g., ECG electrode 26, an electrode on a separate indwelling catheter such as a coronary sinus catheter, and/or the like) that is separate from the electrodes 110 and that is not used to deliver PFA energy to the treatment site may be used to monitor the electrical signal of the heart that is used to generate the ECG. For example, a separate indwelling coronary sinus catheter may be used to measure both atrial and ventricular signals at the same time. In some instances, the electronic processor 205 receives additional or alternative information to monitor a cardiac cycle and associated timing of the patient. For example, the electronic processor 205 may receive intracardiac electrogram (EGM) information and/or other information from one or more other electrodes and/or devices/sensors.

At block 510, the electronic processor 205 of the generator 14 determines, based on the electrical signal monitored at block 505 (e.g., the ECG), an end-diastolic time period (e.g., a ventricular end-diastolic time period 335) at an end of a diastolic time period (e.g., a ventricular diastolic time period 320) during which diastole of the heart (e.g., ventricular diastole) has occurred during a previous cardiac cycle. In some aspects, diastole of the heart may refer to diastole of any one of different specific portions of the heart (e.g., left ventricle, right ventricle, left atrium, right atrium, a specific portion of one of the previous general portions of the heart such as an apex, a base, a ventricular septum, an atrial septum, or the like). In some aspects, the specific diastolic time period and end-diastolic time period of the heart that is determined at block 510 may be determined differently based on a location of the heart that is the treatment site that is intended to receive PFA energy. For example, in situations where PFA energy is provided to one of the atria instead of to a ventricle, the delivery of PFA energy may be timed in accordance with atrial diastole or an atrial end-diastolic period. For example, the delivery of PFA energy may be timed to be immediately before the contraction of the atria, (i.e., immediately before the P-wave) instead of immediately before the R-wave when PFA energy is being delivered to a ventricle.

The example end-diastolic time period 335 shown in FIG. 3 is a time period in which the thickness of the left ventricular wall is low or at a minimum just before occurrence of the R-wave 310 that starts isometric contraction of the left ventricle. However, in some aspects, the end-diastolic time period is alternatively a different time period in which the thickness of a wall of another portion of the heart (e.g., right ventricle, one of the atria, a specific portion of one of the previous general portions of the heart such as an apex, a base, a ventricular septum, an atrial septum, or the like) is at a minimum. Accordingly, the end-diastolic time period 335 shown in FIG. 3 is an example and may be sized differently and/or shifted within a cardiac cycle depending on a portion of the heart that is the treatment site that is being ablated by receiving PFA energy. For example, based on known characteristics of the heart, graphs of a wall thickness of different portions of the heart may vary from the graph 330 of the left ventricular wall thickness shown in FIG. 3. These graphs (not shown) of wall thickness of different portions of the heart throughout a cardiac cycle (e.g., a graph of atrial wall thickness) may be used in a similar manner as the graph 330 is used, and similar calculations as described below with respect to the end-diastolic time period 335 and the diastolic time period 320 may be made to determine other end-diastolic time periods and diastolic time periods for different portions of the heart. These other end-diastolic time periods may be useful when the treatment site where PFA energy is to be delivered is different than the left ventricle (or is a specific portion of the left ventricle such as the apex or the base).

In some aspects, the electronic processor 205 is configured to determine the end-diastolic time period 335 by determining a first time interval between occurrences of a first wave and a second wave included in the electrical signal (e.g., ECG) of one or more previous cardiac cycles. In some aspects, the first wave and the second wave are successive occurrences of the same first type of wave included in the electrical signal. In other words, in some aspects, the electronic processor 205 is configured to determine a first time interval between successive occurrences of a first type of wave included in the electrical signal.

For example, the first type of wave may be an R-wave 310 and the first time interval may be an RR interval 340 between peaks of successive R-waves 310 of successive cardiac cycles as shown in FIG. 3. As another example, the first type of wave may be a P-wave 345 and the first time interval may be a PP interval. As an example where the first wave and the second wave are not the same type of wave, the first wave may be a P-wave 345 and the second wave may be an R-wave 310. In this example, the first time interval may be a PR interval 350 between a peak of a P-wave 345 and the next R-wave 310 as shown in FIG. 3. Other types of waves and intervals may also be used in some aspects. In some aspects, the first wave, the second wave, and/or the first type of waves are selected based on a location of the heart that is intended to receive the PFA energy. For example, the electronic processor 205 may be programmed to detect different waves in the ECG and/or different time intervals between waves in the ECG based on a location of the heart that is intended to receive the PFA energy. As a more specific example, while timing delivery of PFA energy to be immediately before a peak of an R-wave 310 as explained below may be useful when the PFA energy is delivered to one of the ventricles, timing delivery of PFA energy to be immediately before a peak of a P-wave 345 using similar techniques may be useful when the PFA energy is delivered to one of the atria. Accordingly, for atrial applications, instead of determining an RR interval 340 or a PR interval 350, the electronic processor 205 may determine a PP interval or an RP interval.

In some aspects, the electronic processor 205 is configured to determine the first time interval by determining an average of a plurality of measured time values corresponding to the first interval (e.g., from multiple cardiac cycles). While time intervals in a heart's ECG (e.g., RR intervals 340, PR intervals 350, etc.) may remain somewhat consistent between cardiac cycles, some variability may exist in these time intervals when comparing different cardiac cycles. Since the method 500 is designed to deliver PFA energy to a treatment site of the heart at a very specific time during the cardiac cycle, in some aspects, consistency of measured time values corresponding to a time interval in the cardiac cycle is determined by the electronic processor 205. For example, the electronic processor 205 may be configured to determine a time value for each of a plurality of first time intervals included in the electrical signal over an evaluation time period including multiple cardiac cycles before the PFA energy is delivered in a later cardiac cycle. The electronic processor 205 may also be configured to determine a variation between the time values and compare the variation to a variation threshold. For example, the variation threshold may be a standard deviation value or a difference between a highest measured time value and a lowest measured time value included in the plurality of first time intervals. In response to determining that the variation is below the variation threshold, the electronic processor 205 may be configured to establish the first time interval by determining an average of the time values of the plurality of first time intervals. In some aspects, determining that the variation is below the variation threshold indicates that the time values of the plurality of first time intervals are consistent enough to be accurately used to time delivery of PFA energy. In response to determining that the variation is greater than or equal to the variation threshold, the electronic processor 205 may be configured to continue monitoring the electrical signal (at block 505) and/or may provide a notification to a user.

Because the consistency of the time values of the plurality of first time intervals impacts the accuracy of the timing of the delivery of PFA energy, in some aspects, the heart may be artificially paced by one or more of the electrodes 110 or by a separate pacing device. In some aspects, the evaluation period during which the plurality of first time intervals is measured before the PFA energy is delivered may be configured to be longer when the heart of the patient is not artificially paced than when the heart of the patient is artificially paced. For example, when the heart is artificially paced, the evaluation time period to attempt to ensure consistent time intervals within cardiac cycles may be lower than when the heart is not artificially paced since cardiac cycles of an artificially paced heart will likely be more consistent than cardiac cycles of a heart that is not artificially paced.

In some aspects, the electronic processor 205 determines a time range (e.g., an average time range) of the end-diastolic time period 335 during one or more previous cardiac cycles based on the average value of the plurality of first time intervals that was determined as explained previously herein. For example, the electronic processor 205 may be configured to determine that the end-diastolic time period 335 occurs during a range of 90% to 99% (or 80% to 99% or 70% to 99% or 60% to 99% or 50% to 99% or 90% to 95% or 80% to 95%) of the first time interval (e.g., RR interval 340) between successive occurrences of the first type of wave (e.g., R-wave 310) after an instance of the first type of wave (e.g., R-wave 310). Continuing this example, if the average value of the first time interval is 1000 milliseconds, then the end-diastolic time period 335 may be determined to be 900 milliseconds to 990 milliseconds. As another example, the electronic processor 205 determines that the end-diastolic time period 335 occurs during a range of 50% to 99% (or 30% to 99% or 1% to 99%) of the first time interval (e.g., PR interval 350) between occurrences of the first wave (e.g., P-wave 345) and the second wave (R-wave 310) after an instance of the first wave (e.g., P-wave 345). As indicated in FIG. 3 and by the above examples, the electronic processor 205 may be configured to determine the end-diastolic time period 335 as a time period immediately before another instance (e.g., the next predicted instance) of the first wave or first type of wave based on monitoring of previous cardiac cycles. Accordingly, during at least a portion of the end-diastolic time period 335, a myocardium/heart wall thickness is expected to be low or at a minimum as shown in FIG. 3, for example, just before the left ventricle begins to contract to force blood out of the left ventricle.

At block 515, the electronic processor 205 is configured to determine, based on the electrical signal (e.g., the ECG), that another cardiac cycle has begun. In some instances, the electronic processor 205 is configured to determine that each cardiac cycle begins in response to determining another instance of an occurrence of the first wave or the first type of wave in the electrical signal. For example, the beginning of a cardiac cycle may be determined by the electronic processor 205 sensing an R-wave 310, a P-wave 345, or another type of wave. In response to detecting another instance of a wave of the same type, the electronic processor 205 may determine that the previous cardiac cycle has ended and that a new cardiac cycle has begun. For example, the electronic processor 205 may determine a PR interval 350 (at block 510) and may use each detection of a P-wave 345 to indicate that a new (i.e., another) cardiac cycle has begun even though the P-wave 345 may be considered, in some circumstances, to occur in the middle of a cardiac cycle. As another example, the electronic processor 205 may determine an RR interval 340 (at block 510) and may use each detection of an R-wave 310 to indicate that a new (i.e., another) cardiac cycle has begun. In some instances, a start point, an end point or both that start point and the end point of the intervals described herein (e.g., the RR interval 340, the PR interval 350, etc.) may be defined by the time when respective waves that make up the interval is at a peak value (e.g., see the RR interval 340 labeled in FIG. 3) because peak values of respective waves may be easiest to detect in monitored data. In some instances, a start point, an end point, or both of the start point and the end point of the intervals described herein may be defined by the time when respective waves that make up the interval are just beginning (e.g., see the beginning of the PR interval 350 labeled in FIG. 3).

At block 520, the electronic processor 205 causes one or more of the electrodes 110 to deliver pulsed field ablation (PFA) energy to the heart during at least a portion of a time during which the end-diastolic time period 335 of the another (i.e., newly detected and current) cardiac cycle is expected to occur. For example, the electronic processor 205 controls generation of waveforms/signals provided to the electrode(s) 110 to cause the electrode(s) 110 to provide/deliver PFA energy to the heart during at least a portion of a time during which the end-diastolic time period 335 of the another (i.e., newly detected and current) cardiac cycle is expected to occur. In some instances, the end-diastolic time period 335 of the another cardiac cycle may be expected to occur at approximately the same time that the end-diastolic time period 335 occurred in one or more previous cardiac cycles. In other words, the electronic processor 205 may be configured to determine when the end-diastolic time period 335 of the another cardiac cycle is expected to occur based when the end-diastolic time period 335 occurred during one or more previous cardiac cycles. In some instances, the PFA energy may be delivered at any point in time during the end-diastolic time period 335 or over any time period during the end-diastolic time period 335 (e.g., during when the end-diastolic time period 335 is expected to be occurring). In some instances, the PFA energy may be delivered as close to the end of the end-diastolic time period 335 (e.g., as close to estimated time of the next R-wave 310) as possible to attempt to ensure that a myocardium/heart wall thickness is as low as possible or at a minimum. In some instances, the PFA energy may be delivered to the heart during at least a portion of a time in which the end-diastolic time period 335 of the current cardiac cycle is expected to occur based on the first time interval determined based on previous cardiac cycles (at block 510). For example, PFA energy is delivered to the heart during at least a portion of a time included in a range of 90% to 99% (or one of the other ranges listed above or similar ranges) of the first time interval after the another instance of the first type of wave occurred because this range corresponds to a time period during which the end-diastolic time period 335 of the another/current cardiac cycle is expected to occur. In some instances, the PFA energy is delivered during a Q-wave 355 and/or during a beginning part of an R-wave 310 before a peak of the R-wave 310 is detected (as indicated by the end-diastolic time period 335 shown in FIG. 3).

As indicated by the above explanation, execution of the method 500 allows the electronic processor 205 to control timing of delivery of PFA energy to a treatment site of a heart such that the PFA energy is delivered at a low or minimum myocardium/heart wall thickness of the treatment site without actually/physically measuring the myocardium/heart wall thickness of the treatment site.

As indicated in FIG. 5, after block 520 is executed, the method 500 may proceed back to block 505 and may repeat.

In some instances, an imaging device (e.g., an echocardiogram imaging device, magnetic resonance imaging (MRI) device, computed tomography (CT) scanning device, etc.) may be used concurrently and in combination with monitoring of the electrical signal (e.g., the ECG) of the heart before the PFA energy is delivered in order to determine a time period during the heart beat during which a low or minimum myocardium/heart wall thickness of the treatment site occurs. For example, using images (e.g., ventriculograms) obtained by the imaging device (e.g., a transesophageal echocardiogram device, an intracardiac echocardiogram device, and/or the like), a low or minimum myocardium/heart wall thickness of the treatment site may be determined by the electronic processor 205. The images may be time stamped such that the electronic processor 205 (which also may be monitoring the electrical signal of the heart) may determine a time period during the heartbeat (e.g., the same/similar time period during a plurality of heartbeats) during which a low or minimum myocardium/heart wall thickness of the treatment site occurs. The electronic processor 205 may then determine the end-diastolic time period 335 during which PFA energy is applied to the treatment during later heart beats based on the combined imaging/electrical signal information (e.g., during a time period of the heartbeat when the images indicate that the myocardium/heart wall thickness of the treatment site is at a low or minimum). In some instances, the electronic processor 205 may be configured to determine one or more other time periods during the heart beat during which the PFA energy may be applied (e.g., due to low or minimum heart wall thickness) as long as such other time periods do not conflict with other studies, determinations, etc. regarding when the PFA energy may be safely applied.

Accordingly, in some instances, timing of the delivery of the PFA energy to the heart may be determined on a patient specific basis of when a low or minimum myocardium/heart wall thickness of the treatment site occurs. Such timing may be determined using (i) timing estimates from previous cardiac cycles and/or (ii) thickness measurements based on images taken during previous cardiac cycles.

The ranges included herein (e.g., the percentage ranges of the first time interval) are examples. One or both ends of each of these example ranges may vary by, for example, 1%, 5%, 10%, etc. These example ranges are intended to delineate an approximate time range during which a myocardium/heart wall thickness of the treatment site is estimated/expected to be low or at a minimum thickness compared to the myocardium/heart wall thickness at other times in a cardiac cycle.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method of ablating cardiac tissue, the method comprising:
monitoring an electrical signal of a heart of a patient, the electrical signal representing the heart beating;
determining, with an electronic processor and based on the electrical signal, an end-diastolic time period at an end of a diastolic time period during which diastole of the heart has occurred during a previous cardiac cycle;
determining, with the electronic processor and based on the electrical signal, that another cardiac cycle has begun; and
causing, with the electronic processor, an electrode to deliver pulsed field ablation (PFA) energy to the heart during at least a portion of a time in which the end-diastolic time period of the another cardiac cycle is expected to occur.

2. The method of claim 1, wherein determining the end-diastolic time period includes determining, with the electronic processor and based on the electrical signal, a first time interval between occurrences of a first wave and a second wave included in the electrical signal of one or more previous cardiac cycles;
wherein determining that the another cardiac cycle has begun includes determining, with the electronic processor and based on the electrical signal, that another instance of the first wave has occurred in a current cardiac cycle; and
wherein causing the electrode to deliver the PFA energy to the heart includes causing, with the electronic processor, the electrode to deliver the PFA energy to the heart during the at least a portion of the time in which the end-diastolic time period of the current cardiac cycle is expected to occur based on the first time interval.

3. The method of claim 2, wherein the first wave and the second wave are a same type of wave, and wherein the first time interval occurs between successive occurrences of a first type of wave included in the electrical signal.

4. The method of claim 3, wherein the first time interval includes an RR interval and the first type of wave includes an R-wave.

5. The method of claim 3, further comprising selecting a type of the first type of wave based on a location of a treatment site of the heart that is intended to receive the PFA energy.

6. The method of claim 2, wherein the first wave is a first type of wave and the second wave is a second type of wave that is different than the first type of wave.

7. The method of claim 6, wherein the first type of wave includes a P-wave and the second type of wave includes an R-wave, and wherein the first time interval includes a PR interval.

8. The method of claim 6, further comprising selecting the first type of wave, the second type of wave, or both based on a location of a treatment site of the heart that is intended to receive the PFA energy.

9. The method of claim 2, wherein determining the first time interval includes:
determining a time value for each of a plurality of first time intervals included in the electrical signal over an evaluation time period before the PFA energy is delivered;
determining a variation between the time values;
comparing the variation to a variation threshold; and
in response to determining that the variation is below the variation threshold, establishing the first time interval by determining an average of the time values.

10. The method of claim 9, wherein the evaluation time period is longer when the heart of the patient is not artificially paced than when the heart of the patient is artificially paced.

11. The method of claim 2, further comprising determining, with the electronic processor, that the end-diastolic time period is expected to occur during a time in a range of 90% to 99% of the first time interval after the another instance of the first type of wave occurred.

12. The method of claim 1, wherein the diastole of the heart includes one of diastole of a left ventricle, diastole of a right ventricle, diastole of a left atrium, and diastole of a right atrium.

13. The method of claim 1, wherein the end-diastolic time period of the another cardiac cycle indicates that a treatment site of the heart that is intended to receive the PFA energy includes myocardium that has a minimum thickness, compared to a thickness of the myocardium throughout the rest of the another cardiac cycle, during at least a portion of the time in which the end-diastolic time period of the another cardiac cycle is expected to occur.

14. A system for ablating cardiac tissue, the system comprising:
a generator including an electronic processor configured to
monitor an electrical signal of a heart of a patient, the electrical signal representing the heart beating,
determine, based on the electrical signal, an end-diastolic time period at an end of a diastolic time period during which diastole of the heart has occurred during a previous cardiac cycle,
determine, based on the electrical signal, that another cardiac cycle has begun, and
cause an electrode to deliver pulsed field ablation (PFA) energy to the heart during at least a portion of a time in which the end-diastolic time period of the another cardiac cycle is expected to occur.

15. The system of claim 1, wherein the electronic processor is configured to determine the end-diastolic time period by determining, based on the electrical signal, a first time interval between occurrences of a first wave and a second wave included in the electrical signal of one or more previous cardiac cycles;
wherein the electronic processor is configured to determine that the another cardiac cycle has begun by determining, based on the electrical signal, that another instance of the first wave has occurred in a current cardiac cycle; and
wherein the electronic processor is configured to cause the electrode to deliver the PFA energy to the heart by causing, the electrode to deliver the PFA energy to the heart during the at least a portion of the time in which the end-diastolic time period of the current cardiac cycle is expected to occur based on the first time interval.

16. The system of claim 15, wherein the electronic processor is configured to determine that the end-diastolic time period is expected to occur during a time in a range of 90% to 99% of the first time interval after the another instance of the first type of wave occurred.

17. The system of claim 14, wherein the diastole of the heart includes one of diastole of a left ventricle, diastole of a right ventricle, diastole of a left atrium, and diastole of a right atrium.

18. The system of claim 14, wherein the end-diastolic time period of the another cardiac cycle indicates that a treatment site of the heart that is intended to receive the PFA energy includes myocardium that has a minimum thickness, compared to a thickness of the myocardium throughout the rest of the another cardiac cycle, during at least a portion of the time in which the end-diastolic time period of the another cardiac cycle is expected to occur.

19. A method of ablating cardiac tissue, the method comprising:
monitoring an electrical signal of a heart of a patient, the electrical signal representing the heart beating;
determining, with an electronic processor and based on the electrical signal, a first time interval between occurrences of a first wave and a second wave included in the electrical signal of one or more previous cardiac cycles;
determining, with the electronic processor and based on the electrical signal, that another instance of the first wave has occurred in a current cardiac cycle; and
causing, with the electronic processor, an electrode to deliver pulsed field ablation (PFA) energy to the heart during at least a portion of a time included in a range of 90% to 99% of the first time interval after the first wave has occurred in the current cardiac cycle.

20. The method of claim 19, wherein the time included in the range of 90% to 99% of the first time interval after the first wave has occurred indicates that a treatment site of the heart that is intended to receive the PFA energy includes myocardium that has a minimum thickness, compared to a thickness of the myocardium throughout the rest of the current cardiac cycle, during at least a portion of the time included in the range of 90% to 99% of the first time interval after the first wave has occurred.
